(19) United States
(12) Patent Application Publication
(10) Pub. No.: US 2025/0362309 A1
(43) Pub. Date: Nov. 27, 2025

(54) BIOMARKERS AND USE THEREOF FOR DIAGNOSIS, PREVENTION, AND TREATMENT OF MUSCLE ATROPHY

(71) Applicant: MYOMAR MOLECULAR INC., Bedford (CA)

(72) Inventors: Rafaela P.L. De Andrade Vieira, Bedford (CA); Dylan Deska-Gauthier, Bedford (CA)

(73) Assignee: MYOMAR MOLECULAR INC., Bedford, NS (CA)

(21) Appl. No.: 18/874,552

(22) PCT Filed: Jun. 13, 2023
(86) PCT No.: PCT/CA2023/050810

§ 371 Date: Dec. 12, 2024

Related U.S. Application Data
(60) Provisional application No. 63/351,591, filed on Jun. 13, 2022.

(51) Int. Cl. G01N 33/68

(52) U.S. Cl. CPC G01N 33/6893; G01N 33/6806; G01N 2800/10; G01N 2800/52

(57) ABSTRACT

There is provided a method for diagnosing a subject with early onset muscle atrophy, said method comprising: obtaining a biosample from the subject; and assaying the biosample for one or more biomarkers, said one more biomarkers are taurine, proline, citrulline, trigonelline, thymidine, ornithine, glutamate, L-pyroglutamic acid, creatinine, adenine, nicotinamide, 2-methylhippuric acid, maltol, L-arginine, hypotaurine, L-glutamine, homogentisic acid, methylhistidine, oxoglutaric acid, xanthine, L-carnitine, succinate, or a combination thereof; and identifying the subject with early onset muscle atrophy on the basis of a deviation in the one or more of said one more biomarkers.

### CROSS REFERENCE TO RELATED APPLICATION

The application claims the benefit of priority to U.S. Provisional Application No. 63/351,591 filed Jun. 13, 2022 and entitled BIOMARKERS AND USE THEREOF FOR DIAGNOSIS, PREVENTION, AND TREATMENT OF MUSCLE ATROPHY, the contents of which are herein incorporated by reference in its entirety.

### TECHNICAL FIELD

The present invention relates to biomarkers and use thereof in systems, kits, methods of and use for diagnosis, prevention, and treatment of muscle atrophy.

### BACKGROUND

Skeletal muscle regeneration is essential to maintain muscle integrity and function. Muscle metabolism is directly linked to muscle regeneration and maintenance. Homeostasis of muscle metabolism is very important to maintain muscle function and an unbalance of this process leads to loss of muscle mass which correlates with a number of muscle related disorders in addition of aging.

Physical examination is currently the most commonly used method for accessing muscle weakness/health and recovery states. Physical examination is carried out by professionals, such as physiotherapists, however this type of examination is subjective and time consuming, and there is a need for alternatives.

Bioimpedance tests do not directly measure muscle mass, and such results are often misinterpreted if patients have high adipose tissue. As well, in the case of obese or frail patients, bioimpedence tests cannot be performed or are impractical. For patients who are in the hospital, a health care provider would measure bioimpedance and albumin levels in order to make decisions about diet and exercise plan. This is also subjective as it does not measure muscle atrophy directly, therefore the health care provider will follow a treatment course that is not specifically targeting muscle health. Another option for bedridden patients is magnetic resonance imaging (MRI) or dual x-ray absorptiometry (DXA) scan which are accurate but very expensive and requires a highly trained professional to perform the analysis. Often orthopedists will use MRI or DXA prior to surgery in order to analyze ortho-related conditions but not specifically for muscle injuries.

Another method for identifying muscle atrophy is through a muscle biopsy, but this method is invasive and not readily accessible. As a consequence, evaluation of muscle loss of bedridden patients are rarely performed. Because of lacking accessible and specific diagnostic tools of muscle atrophy, after discharge, patients will spend months and even years recovering muscle mass that was lost during their hospitalization period. As a further consequence, without knowing muscle loss levels it is difficult to create a rehabilitation program to specifically target muscle loss which would benefit patients' muscle health and quality of life post-hospitalization.

Muscle recovery following knee replacement surgeries cause direct and indirect costs to individuals as well as the health care system and other surrounding institutions. These costs are estimated at $1.4 billion dollars annually in Canada. Osteoarthritis can take weeks to years to manifest in individuals. For aging populations, individuals can enter a state of osteoarthritis that can go undealt with until the deterioration of joint, pain and lack of mobility. At this point, most seniors are directed to knee replacements, which have been a common solution for pain and regaining of independence.

Total knee replacements are a very common procedure in the aging population. In Canada alone, there are over 75 thousand surgeries a year and this number is projected to increase in the next few years because of the growing aging population. This type of surgery greatly benefits quality of life by decreasing pain, allowing physical activity and social interaction. However, on average 30% of patients are dissatisfied with surgery outcomes because they still experience residual pain and cannot return to regular physical activities. In many cases severity of osteoarthritis can predict the outcome of surgery and quality of life. The primary reason for a knee replacement is osteoarthritis, this condition causes progressive degeneration of joints resulting in inflammation, pain, and loss of mobility. People with knee osteoarthritis have increased muscle wasting, or sarcopenia, resulting from intrinsic changes to the joint and reduced physical activity levels. Muscle weakness is a determinant of pain and disability for people living with osteoarthritis. Muscle tissue plays an important role in joint stability and health, and muscle wasting is associated with a greater risk of osteoarthritis. Conversely, muscle training is used as a way to reduce disease progression and is an important target for rehabilitation postoperatively and conservation of health.

Both surgery performance and rehabilitation are crucial to the success of knee replacement surgeries. In particular, rehabilitation practice can be performed pre-operatory or post-operatory. Rehabilitation pre-operatory is key to address early onset disease and even prevent surgeries, however, it is rarely implemented. More recently, there is more interest in pre-rehabilitation programs due to the importance of preventive care and its association with reduced risk of complications post-surgery. Despite this, rehabilitation post-operatory is the most common practice. Rehabilitation postoperatively improves mobility, pain, and quality of life. Further, rehabilitation mitigates the risk of a secondary revision surgery which is costly to the individual and surrounding health care systems. Indeed, the cost of knee replacements are on average $10,000 per patient, while revision surgeries cost an additional $17,000 in Canada.

Therefore, successful post-operative rehabilitation programs are important to improve muscle health which can lower the risk of surgical revisions and their negative consequences. The ability to track rehabilitation success and care of patients that are mostly at-risk of revisions due to comorbidities or severity of osteoarthritis can improve personalized treatment and final outcomes. However, the best way to determine successful rehabilitation postoperatively remains elusive, particularly with respect to regaining muscle structure. Indeed, subjective measures that are time delayed are often used to measure muscle wasting. For example, the gold standard to access physical recovery is based on measurements of strength and range of motion. These measurements take up to 6-8 weeks after the start of rehabilitation to demonstrate changes, thus, it makes it difficult to monitor progress. This practice is subjective as these parameters vary with human behaviors, mental state and often do not reflect recovery success. An objective and simple metric of muscle molecular changes could provide an accurate reflection of muscle recovery over time. However, no such measurement exists to date.

The detection of muscle health could contribute to better prognosis and pre-rehabilitation that would affect surgery outcomes. Following surgery, muscle deterioration is prevalent due to disuse and hospitalization time. Therefore, a healthier muscular state of the individual prior to knee replacements can alleviate this muscle deterioration and prevent complications. In fact, many have shown that pre-rehabilitation programs lead to better surgery outcome and regain of physical activity early on.

After knee replacements, individuals are often dependent on the healthcare system and others around them for long periods of time during their recovery. Therefore, an efficient rehabilitation process is crucial for the regaining of independence and recovery success. The current practice relies on physical assessment to monitor rehabilitation programs. Individuals are routinely subjected to generic physiotherapy during which it may take up to 8 weeks for observable functional changes in response to a therapy regimen. Therefore, there is a need to provide knowledge that will promote the development of a more cost-effective approach that is reliable and more individualized than the current practice.

There is a need for identification of molecular biomarkers to identify recovery in an early stage so health care providers can better monitor and construct more individualized treatment plans. There is a need for a method to diagnose a subject with early onset muscle atrophy to promote pre-rehabilitation processes that can potentially save health care system and individual costs by diverging surgeries, reducing time in the hospital, expediting the rehabilitation process and diminishing revision surgeries to improve quality of life and help to establish preventive measures to mitigate risks of osteoarthritis.

In addition to musculoskeletal diseases, muscle metabolism is critical for neuromuscular diseases (NMD) such as muscular dystrophies, amyotrophic lateral sclerosis and others. Currently, the molecular diagnosis of NMD poses a significant challenge, as it requires specialized training in both modern and conventional techniques with few laboratories having the capability to provide a comprehensive characterization of NMD. Predicting factors of disease onset and severity that are critical for genetic counselling, treatment, and prevention of potential complications remain unknown. Together, these unknown elements orchestrate a significant challenge for disease management, leading to a lack of adequate therapy, delays in correct intervention, and a lack of proactive approaches. NMD prognosis also remains an issue because of the unpredictable manifestations of the disease and the absence of accurate markers. As such, many cases remain subclinical due to the slight and progressive state of the disease.

The degeneration of muscle fibers encountered in musculoskeletal disease is similar across NMDs, and is characterized by the shortening of the fibers which induces fat infiltration, mitochondria metabolism dysregulation, and apoptosis of myocytes.

### SUMMARY OF THE INVENTION

The present application includes biomarkers, uses, methods, devices, reagents, systems, and kits for the detection and diagnosis, prevention, and treatment of muscle atrophy.

In one embodiment, the present invention contemplates biomarkers, uses, methods, devices, reagents, systems, and kits for evaluating muscle atrophy in a subject. As described herein, measurement of biomarkers described herein can be used for diagnosis, prognosis, risk stratification, staging, monitoring, categorizing and a determination of further diagnosis and treatment regimens in subjects suffering or at risk of suffering from muscle atrophy.

In one embodiment, the present invention contemplates biomarkers, uses, methods, devices, reagents, systems, and kits to allow each muscle-related disease subject to continuously track their own muscle health during and after any therapeutic intervention in the case of neuromuscular diseases to improve quality of life and management of muscle-related disease progression.

It is an embodiment of the present invention to provide a method for diagnosing a patient with early onset muscle atrophy, said method comprising: obtaining a biosample from the patient; assaying the biosample for one or more biomarkers, said one more biomarkers are taurine, proline, citrulline, trigonelline, thymidine, ornithine, glutamate, L-pyroglutamic acid, creatinine, adenine, nicotinamide, 2-methylhippuric acid, maltol, L-arginine, hypotaurine, L-glutamine, homogentisic acid, methylhistidine, oxoglutaric acid, xanthine, L-carnitine, succinate, or a combination thereof; identifying the patient with early onset muscle atrophy on the basis of a deviation in the one or more of said one more biomarkers.

It is an embodiment of the present invention to provide a method of identifying a patient with muscle atrophy and treating said patient with muscle atrophy, said method comprising: identifying a suitable patient for treatment, said identifying comprising assaying a biosample obtained from a candidate patient for changes in one or more biomarkers, said one more biomarkers are taurine, proline, citrulline, trigonelline, thymidine, ornithine, glutamate, L-pyroglutamic acid, creatinine, adenine, nicotinamide, 2-methylhippuric acid, maltol, L-arginine, hypotaurine, L-glutamine, homogentisic acid, methylhistidine, oxoglutaric acid, xanthine, L-carnitine, succinate, or a combination thereof; selecting the suitable patient on the basis of a significant change in one or more of said one more biomarkers in the biosample of the candidate patient, wherein said significant change is indicative of muscle atrophy; administering to the suitable patient a rehabilitation regimen selected for increasing muscle growth; identifying the treated patient when there is no longer a significant change in one or more of said one more biomarkers.

It is an embodiment of the present invention to provide a system and method for guiding muscle recovery and/or muscle health maintenance of an individual with early onset muscle atrophy or an individual in need of treatment of muscle atrophy. The method comprising identifying levels of biomarkers of atrophy prior to administering an exercise regimen and/or therapeutic intervention as compared to normal muscle; and identifying changes in the levels of biomarkers of atrophy after the administering an exercise regimen or a therapeutic intervention as compared to the levels prior to the exercise regimen or the therapeutic intervention.

In one aspect, the muscle atrophy is as a result of one or more of age, disuse, sedentary, disease (such as NMD) and physical trauma or injury. In some aspects, physical trauma or injury is as a result of an orthopedic procedure such as for example, joint surgery, and total knee arthroscopy (TKA).

It is an embodiment of the present invention to provide a method of quantifying risk, guiding muscle recovery and muscle health maintenance of a patient after an orthopedic procedure or for the maintenance of physical wellness, the method comprising: obtaining a biosample from the patient prior to the orthopedic procedure or therapeutic intervention in case of muscle-related condition; assaying the biosample for the level of one or more biomarkers to establish a control concentration, said one more biomarkers are taurine, proline, citrulline, trigonelline, thymidine, ornithine, glutamate, L-pyroglutamic acid, creatinine, adenine, nicotinamide, 2-methylhippuric acid, maltol, L-arginine, hypotaurine, L-glutamine, homogentisic acid, methylhistidine, oxoglutaric acid, xanthine, L-carnitine, succinate, or a combination thereof; administering an exercise regimen after the orthopedic procedure; obtaining a further biosample from the patient after and/or during the exercise regimen or the therapeutic intervention; assaying the further biosample for the level of the one or more biomarkers to establish a recovery concentration; comparing the level of the one or more biomarkers differentially produced as a consequence of the exercise regimen or the therapeutic intervention to identify an indication of a reversion to the control concentration from the recovery concentration of said one or more biomarkers; and administering appropriate treatment based on the presence or the absence of the reversion.

It is an embodiment of the present invention to provide a method of quantifying risk, guiding muscle recovery and muscle health maintenance of a patient to preserve and/or maintain physical wellness, the method comprising: obtaining a biosample from the patient; assaying the biosample for the level of one or more biomarkers to establish a control concentration, said one more biomarkers are taurine, proline, citrulline, trigonelline, thymidine, ornithine, glutamate, L-pyroglutamic acid, creatinine, adenine, nicotinamide, 2-methylhippuric acid, maltol, L-arginine, hypotaurine, L-glutamine, homogentisic acid, methylhistidine, oxoglutaric acid, xanthine, L-carnitine, succinate, or a combination thereof; administering an exercise regimen for maintenance of physical health; obtaining a further biosample from the patient after and/or during the exercise regimen; assaying the further biosample for the level of the one or more biomarkers to establish a recovery concentration; comparing the level of the one or more biomarkers differentially produced as a consequence of the exercise regimen to identify an indication of a reversion to the control concentration from the recovery concentration of said one more biomarkers; and administering appropriate treatment based on the presence or the absence of the reversion.

In one aspect, the appropriate treatment is selected from the same exercise regimen or a different exercise regimen and optionally obtaining a further biosample after the same exercise regimen or the different exercise regimen; assaying to establish the recovery concentration; and comparing the level of the one or more biomarkers differentially produced as a consequence of the same exercise regimen or the different exercise regimen to identify an indication of a reversion to the control concentration from the recovery concentration of said one more biomarkers.

It is an embodiment of the present invention to provide a method for diagnosing a subject with early onset muscle atrophy, said method comprising: analyzing a biosample obtained from the subject for one or more biomarkers, said one more biomarkers are taurine, proline, citrulline, trigonelline, thymidine, ornithine, glutamate, L-pyroglutamic acid, creatinine, adenine, nicotinamide, 2-methylhippuric acid, maltol, L-arginine, hypotaurine, L-glutamine, homogentisic acid, methylhistidine, oxoglutaric acid, xanthine, L-carnitine, succinate, or a combination thereof to determine a subject value of said one or more biomarkers; and diagnosing the subject with early onset muscle atrophy where there is a deviation in the subject value from a threshold value of said one or more biomarkers.

It is an embodiment of the present invention to provide a diagnostic system for diagnosing a subject with early onset muscle atrophy, comprising: an analyzing unit for analyzing the biosample for one or more biomarkers including at least one detector for detecting said one or more biomarkers; a database including the threshold value of one or more biomarkers; and an evaluation unit including a computer having a program code for carrying out instructions for diagnosing the subject with early onset muscle atrophy when there is a deviation in the subject value from a threshold value of said one or more biomarkers

It is an embodiment of the present invention to provide a method of identifying a subject with muscle atrophy and treating said subject with muscle atrophy, said method comprising: identifying a suitable subject for treatment, said identifying comprising assaying a biosample obtained from a candidate subject for changes in one or more biomarkers, said one more biomarkers are taurine, proline, citrulline, trigonelline, thymidine, ornithine, glutamate, L-pyroglutamic acid, creatinine, adenine, nicotinamide, 2-methylhippuric acid, maltol, L-arginine, hypotaurine, L-glutamine, homogentisic acid, methylhistidine, oxoglutaric acid, xanthine, L-carnitine, succinate, or a combination thereof to determine a subject value of said one or more biomarkers; selecting the suitable subject on the basis of a deviation to determine of the subject value from a threshold value, wherein said deviation is indicative of muscle atrophy; administering to the suitable subject a rehabilitation regimen selected for increasing muscle growth; and identifying a treated subject when there is no longer a deviation of the subject value from the threshold value.

It is an embodiment of the present invention to provide a method of guiding muscle recovery of a subject suspected of having a neurodegenerative disease, of a subject after an orthopedic procedure, or of a subject to maintain physical health, the method comprising: obtaining a biosample from the subject; assaying the biosample for the level of one or more biomarkers, said one more biomarkers are taurine, proline, citrulline, trigonelline, thymidine, ornithine, glutamate, L-pyroglutamic acid, creatinine, adenine, nicotinamide, 2-methylhippuric acid, maltol, L-arginine, hypotaurine, L-glutamine, homogentisic acid, methylhistidine, oxoglutaric acid, xanthine, L-carnitine, succinate, or a combination thereof to determine a subject value of said one or more biomarkers; administering a therapeutic regimen to the subject suspected of having a neurodegenerative disease, an exercise regimen after the orthopedic procedure or an exercise regimen to maintain physical health; obtaining a further biosample from the subject after and/or during the therapeutic regimen or exercise regimen; assaying the further biosample to establish a recovery value for said one or more biomarkers; comparing the recovery value for said one or more biomarkers differentially produced as a consequence of the therapeutic regimen or exercise regimen to a threshold value to identify an indication of a reversion to the threshold value; and administering any further therapy regimen or exercise regimen based on an absence of the reversion.

It is an embodiment of the present invention to provide a method selecting a therapy to treat muscle atrophy in a subject or evaluating the effect of the therapy in the subject, the method comprising: analyzing a biosample for one or more biomarkers, said one more biomarkers are taurine, proline, citrulline, trigonelline, thymidine, ornithine, glutamate, L-pyroglutamic acid, creatinine, adenine, nicotinamide, 2-methylhippuric acid, maltol, L-arginine, hypotaurine, L-glutamine, homogentisic acid, methylhistidine, oxoglutaric acid, xanthine, L-carnitine, succinate, or a combination thereof, to determine a subject value of said one or more biomarkers before the subject undergoes therapy; identifying a deviation in the subject value from a threshold value; analyzing a further biosample for said one or more biomarkers during or after the therapy when there is a deviation in the subject value from a threshold value; and selecting the therapy as effective for treating early onset muscle atrophy when there is no longer a deviation in the subject value from a threshold value of said one or more biomarkers.

It is an embodiment of the present invention to provide a kit for diagnosing a subject with early onset muscle atrophy, the kit comprising a capture reagent sufficient for binding to one or more biomarkers obtained from a biosample of the subject and capable of producing a quantitative and/or a qualitative detectable signal when bound to said one or more biomarkers, said one more biomarkers are taurine, proline, citrulline, trigonelline, thymidine, ornithine, glutamate, L-pyroglutamic acid, creatinine, adenine, nicotinamide, 2-methylhippuric acid, maltol, L-arginine, hypotaurine, L-glutamine, homogentisic acid, methylhistidine, oxoglutaric acid, xanthine, L-carnitine, succinate, or a combination thereof.

In one aspect, the correlation of the disclosed biomarkers and panel of biomarkers with muscle tissue regeneration in response to rehabilitation programs offers many advantages to the individual and the healthcare system. First, at the individual level the biomarkers provide an accurate and timely reflection of disease severity and patients recovery rate, indicating to care providers whether the patient's rehabilitation program needs to be modified. Second, objective measurement of muscle recovery postoperatively can identify patients who are not recovering as expected and are at risk for a revision surgery. Third, for muscle-related disease it will inform on appropriate therapeutical effectiveness and avoid therapies that are not influencing positively muscle recovery. In summary, objective values of muscle health can provide justification for further therapy or support discharge from rehabilitation program and allow for preservation and/or muscle health maintenance.

In one aspect, the present disclosure will also benefit individuals that are mostly at risk of potential side effects that are more reflective of disease activity minimizing the risk of revision surgeries and ineffective therapy. Current status of muscle health can support pre-rehabilitation programs, targeting reversible stages of the disease when physical deterioration cannot be conclusive. In addition, the obtained information on muscle wasting levels will be useful for determining and/or assessing muscle health for lessening the risk of further musculoskeletal conditions such as muscle injuries, osteoporosis and sarcopenia.

### BRIEF DESCRIPTION OF THE DRAWINGS

FIG. 1. Skeletal muscle biopsy. Immunohistochemistry analysis of myosin expression in lower limb skeletal muscle. A. Fibers were stained with DAPI and WGA to visualize nuclei and cellular membranes, respectively. Myosin staining is detecting heavy chain myosin of newly formed fibers. Graph analysis corresponds to statistics analysis of cross-sectional area (CSA) of myofibers, reduction of CSA corresponds to muscle atrophy states (** p<0.004). B. Electron microscopy of skeletal muscle fibers. Fibers were imaged and ultra-structures were observed. Arrows show z-line of sarcomeres, breaks of Z-lines are highly related to muscle weakness and atrophy. Atrophic tissues have high number of breaks and were quantified in the correspondent graphic (**p<0.001);

FIG. 2. Locomotion and strength. A. Mice were recorded while running on a treadmill for locomotion and kinematic gate analysis. Graphic shows differences in gate patterns between (WT) and NCK1 knockout (KO). B. Grip forces were also measured in these animals and showed decreased strength for atrophic animals (NCK1 KO) compared to normal animals (*p<0.01);

FIG. 3. Metabolomic profile of sedentary versus exercised animals. A. Heat map showing mean of a total of 3 animals, mass spectrometry findings are represented by R1 and R2 (repetition 1 and 2) of the same samples. A specific number of metabolites were altered in sedentary animals (WT vs NCK1 KO) and no alterations were observed after exercise (Table 1). Groups are as followed: A—WT sedentary, B—NCK1 KO sedentary, C—WT exercised and D—NCK1 KO exercised;

FIG. 4. Principal component analysis. Analysis of serum from tissue bank tissue. The international physical assessment questionnaire (IPAQ) was used to determine physical activity levels. Group A;E age 35-45 y high IPAQ, group B;F age 50-70 y high IPAQ, group C;G age 50-70 y low IPAQ and group D;H age 50-7 y low IPAQ plus arthritis and/or osteoporosis. A. Analysis of all metabolites found in male samples. B. Analysis of all metabolites in female samples;

FIG. 5. Metabolomics analyses of group B and C of serum samples. A. Heat map showing mean analysis of groups. B. Principal component analysis of the same metabolites;

FIG. 6. Graph shows metabolites of interest in male samples. Results show peak intensity values (mean+/−SD) of groups B and C, normalization shows −log 10. Statistics analyses were completed by t-test, p values are displayed in the graphs;

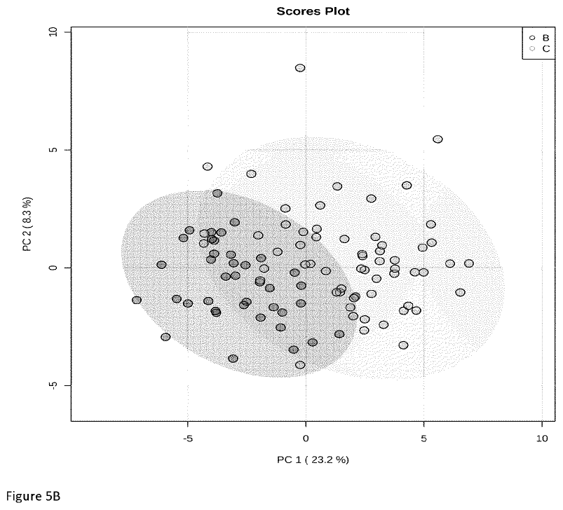

FIG. 7. Metabolomics analyses of group F and G of serum samples. A. Heat map showing mean analysis of groups. B. Principal component analysis of the same metabolites;

FIG. 8. Graph shows metabolites of interest in female samples. Results show peak intensity values (mean+/−SD) of groups F and G, normalization shows −log 10. Statistics analysis were completed by t-test, p values are displayed in the graphs;

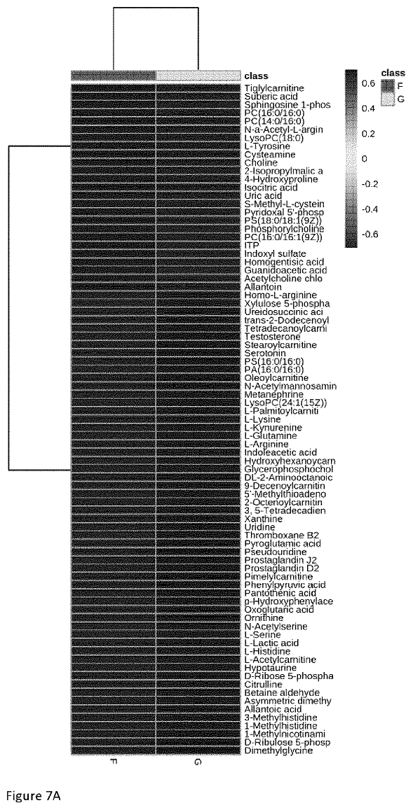

FIG. 9. Identification of metabolites in urine samples and concentration of metabolites using mass spectrometry screen using enzymatic assays;

FIG. 10. Receiver operating curve (ROC) with an area under the curve (AUC) of 0.8 for males and 0.96 for females to show accuracy of the biomarker panel in correlation with physical activity level;

FIG. 11 is a flow chart for a clinical study to correlate biomarkers of muscle atrophy in method of prognosis for aging adults and as evidence-based models to assess muscle health;

FIG. 12. Receiver operating curve (ROC) with an area under the curve (AUC) where FIG. 12A is ALMI and 5× sit and resulted in an AUC score of 0.88; where FIG. 12B shows the combination of SPPB with ALMI and resulted in an AUC score of 0.81; and where FIG. 12C shows the combination of 5× Sit and stand with SPPB resulted in an AUC score of 0.68; and

FIG. 13 depicts the relationship between muscle fiber metabolism and metabolites from urine samples.

### DETAILED DESCRIPTION

This invention is more particularly described below and the Examples set forth herein are intended as illustrative only, as numerous modifications and variations therein will be apparent to those skilled in the art. As used in the description herein and throughout the claims that follow, the meaning of “a”, “an”, and “the” includes plural reference unless the context clearly dictates otherwise. The terms used in the specification generally have their ordinary meanings in the art, within the context of the invention, and in the specific context where each term is used. Some terms have been more specifically defined below to provide additional guidance to the practitioner regarding the description of the invention.

The terms “biomarker” as used herein refer to one or a plurality of molecules, compound, or metabolites identified in a biological sample by the inventive methods related to muscle atrophy. Metabolic signatures and biomarker profiles according to the invention can provide a molecular “fingerprint” of disorder and identify one or preferably a population of cellular metabolites significantly altered in individuals with the disorder. In preferred embodiments, the concentration of the biomarker in said sample may be indicative of a pathological state, metabolic signatures or biomarker profiles are used for diagnosis, prevention, and directing treatment of muscle atrophy in an individual.

The term “metabolite”, as used herein refers to a compound produced or consumed in the metabolism of the subject. A metabolite encompasses all classes of organic or inorganic compounds and may comprise stereoisomers or enantiomers of a compound.

In aspects the disclosed individual biomarkers are useful for detecting and diagnosing muscle atrophy, methods are also described herein for the grouping of multiple subsets of the muscle atrophy biomarkers, where each grouping or subset selection is useful as a panel of three or more biomarkers, interchangeably referred to herein as a “biomarker panel” and a panel. Thus, in some embodiments of the instant application provide combinations comprising one or more biomarkers. In some aspects, the methods comprise a combination of biomarkers linked to muscle loss are useful for detecting and diagnosing muscle atrophy.

The terms “biological sample”, “samples” or “biosamples” include but are not limited to a bodily fluid such as urine, whole blood, blood plasma, serum, sweat, or saliva.

As used herein, “biomarker value”, “value”, “biomarker level”, and “level” are used interchangeably to refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or corresponding to the biomarker in the biological sample. The exact nature of the “value” or “level” depends on the specific design and components of the particular analytical method employed to detect the biomarker.

When a biomarker indicates or is a sign of an abnormal process or a disease or other condition in an individual, that biomarker is generally described as being either over-expressed or under-expressed as compared to a reference which is an expression level or value of the biomarker that indicates or is a sign of a normal process or an absence of a disease or other condition in an individual.

Further, a biomarker that is either over-expressed or under-expressed can also be referred to as being “differentially expressed” or as having a “differential level” or “differential value” as compared to a “normal” expression level or value of the biomarker that indicates or is a sign of a normal process or an absence of a disease or other condition in an individual. Thus, “differential expression” of a biomarker can also be referred to as a variation from a “normal” expression level of the biomarker.

The term “biomarker panel” as used herein refers to a plurality of metabolites. In certain embodiments, the expression levels of the metabolites in the panels can be correlated with the existence of condition of muscle of a subject.

The term “correlation” and “correlating” as used herein, in reference to the use of biomarkers, refers to comparing the presence and/or amount of any biomarker(s) in a subject to its presence and/or amount in persons known to suffer from, or known to be at risk of, a given condition; or in subjects known to be free of a given condition. Often, this takes the form of comparing an assay result in the form of a biomarker concentration to a predetermined threshold selected to be indicative of the occurrence or nonoccurrence of a disease or the likelihood of some future outcome.

The term “subject” or “patient” as used herein, refers to a human or non-human organism. Thus, the methods and compositions described herein are equally applicable to both human and veterinary disease. Preferred subjects or patients are humans that are receiving medical care for a disease or condition.

The term “diagnosis” as used herein, refers to methods by which trained medical personnel can estimate and/or determine the probability (i.e., for example, a likelihood) of whether or not a patient is suffering from a given disease or condition. In the case of the present invention, “diagnosis” includes correlating the results of an assay (i.e., for example, an immunoassay) for a biomarker or a panel of biomarkers of the present invention, optionally together with other clinical indicia (e.g. exercise stress tests), to determine the occurrence or nonoccurrence of an injury or muscle atrophy for a subject or patient from which a sample was obtained and assayed. That such a diagnosis is “determined” is not meant to imply that the diagnosis is 100% accurate. Thus, for example, a measured biomarker level below a predetermined diagnostic threshold may indicate a greater likelihood of the occurrence of a disease in the subject relative to a measured biomarker level above the predetermined diagnostic threshold may indicate a lesser likelihood of the occurrence of the same disease.

The term “prognosis” as used herein, refers to a probability (i.e., for example, a likelihood) that a specific clinical outcome will occur. For example, a level or a change in level of a prognostic indicator, which in turn is associated with an increased probability of morbidity (e.g., worsening muscular function).

In one embodiment, biomarker detection can be achieved using a mass spectrometry (MS)-based method as well as MS-based methods coupled with a separation technique, such as liquid chromatography (LC-MS), known in the art.

The method includes the measurement of at least one metabolite as a specific biomarker for muscle atrophy from a biological sample. Preferably, the level of at least two or more biomarkers is determined to screen or diagnose muscle atrophy, for example, the level of between two to fifteen biomarkers as part of a panel of metabolites to enhance sensitivity and specificity.

In addition to the quantitation of selected biomarkers, a ratiometric determination of two biomarkers may be calculated, i.e. the ratio of the levels of two biomarkers from a sample, for comparison against a control value, i.e. the ratio of the control levels of the two selected biomarkers.

A variety of methods may be used to arrive at a desired threshold value for use in these methods. For example, a threshold value may be determined from a population of normal subjects by selecting a biomarker concentration representing the 75^{th}, 85^{th}, 90^{th} 95^{th}, or 99^{th} percentile of the biomarker as measured in such normal subjects. Alternatively, a threshold value may be determined from a “diseased” population of subjects, e.g., those suffering from an injury or disease (e.g. osteoarthritis), by selecting a biomarker concentration representing the 75^{th}, 85^{th}, 90^{th}, 95^{th}, or 99^{th} percentile of the biomarker as measured in such diseased subjects. In another alternative, the threshold value may be determined from a prior measurement of a biomarker in the same subject; that is, a temporal change in the level of the biomarker in the subject may be used to assign risk to the subject.

The foregoing discussion is not meant to imply, however, that biomarkers contemplated herein are limited to a comparison to corresponding individual thresholds. Other methods for combining assay results can comprise the use of a composite result which is determined by combining individual biomarkers may be treated as if it is itself a biomarker; that is, a threshold value may be determined for the composite result as described herein for individual biomarkers, and the composite result for an individual patient compared to this threshold value. In this way, a receiver-operating characteristic curve (AUC ROC) can be generated to assess the prediction accuracy of each biomarker. A threshold value is selected to provide an acceptable level of specificity and sensitivity where a perfect test will have an area under the ROC curve of 1.0; a random test will have an area of 0.5. Therefore, one or more of the following results include: a specificity of greater than 0.5, preferably at least 0.6, more preferably at least 0.7, still more preferably at least 0.8.

In some embodiments, the present invention also contemplates assay devices for performing the methods described herein. Suitable assay devices comprise capture reagents sufficient for (i.e. capable of binding to a metabolite) performing an assay for at least one of the described biomarkers. In certain embodiments, such assay devices may be included in a kit, together with instructions for performing the assay. Exemplary capture reagents can comprise one or more solid phase antibodies, the solid phase antibody comprising antibody that detects the intended biomarker target(s) bound to a solid support. In the case of sandwich immunoassays, such reagents can also include one or more detectably labeled antibodies, the detectably labeled antibody comprising antibody that detects the intended biomarker target(s) bound to a detectable label. In some embodiments, the kit includes instructions for using the kit and is a lateral flow test, chemiluminescence immunoassay, chromatographic assay or fluorescence immunoassay.

In some embodiments, the present invention also contemplates a diagnostic system for diagnosing a subject with early onset muscle atrophy. The system comprising analyzing unit for analyzing the biosample for one or more metabolites. The analyzing unit including at least one detector for detecting the one or more biomarkers. For example, where the detector allows for automatic qualitative or quantitative determination of the biomarker, the data obtained by said automatically operating analyzing unit can be processed by, e.g., a computer program in order to facilitate the assessment in the evaluation unit. Preferably, the evaluation unit includes a data processing device for processing the resulting data for the assessment and for establishing the output information and a data collection unit comprises values of all diagnostic biomarkers.

Embodiments of the invention are described in the following examples which are not to be construed as limiting.

### Examples

### Example 1—Metabolic Profiling of NCK1 Knockout (KO) Animals

Skeletal muscle regeneration is essential to maintain muscle integrity and function. Muscle metabolism is directly linked to muscle regeneration and maintenance. Homeostasis of muscle metabolism is very important to maintain muscle function and an unbalance of this process leads to loss of muscle mass which correlates with a number of muscle related disorders in addition of aging.

Upon myofibril injury, there is an activation of muscle stem cells progenitors that are capable to proliferate and differentiate into mature myocytes to regenerate the damaged tissues establishing myogenesis integrity pathways. Adaptor proteins play an important role serving as chaperones bidding to proteins and promoting downstream signaling. These proteins are also linked to cellular remodeling through their role in actin dynamics. NCK1, an adaptor protein, is involved in cellular signaling leading to actin remodeling and cell growth.

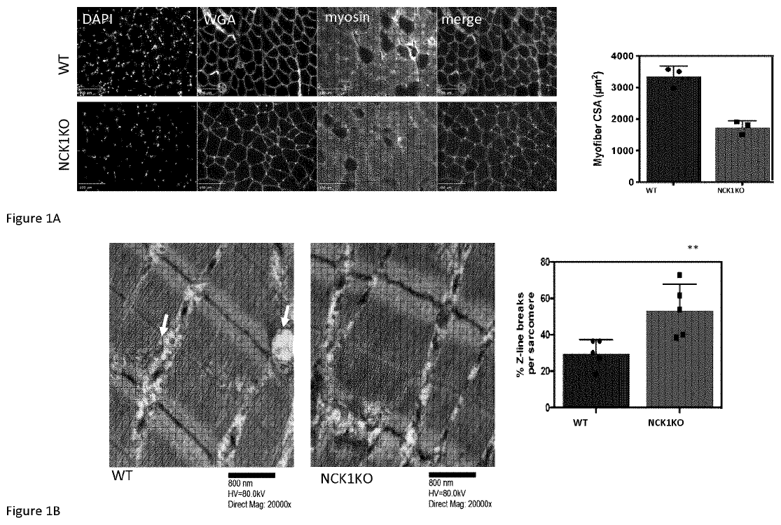

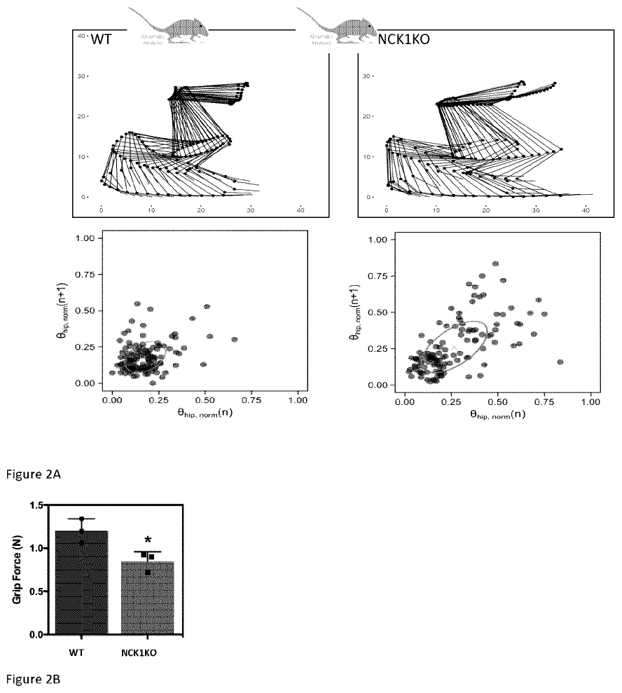

NCK1 knockout (KO) animals have abnormal muscle formation with early signs of muscle atrophy and deterioration of sarcomeres compared to wild type (WT) (FIGS. 1A and B). WT were composed of animals with homogenous fiber sizes with no signs of muscle atrophy nor muscle weakness measured by grip strength. NCK1 KO animals have decreased grip strength compared to WT (FIGS. 2A and 2B).

The metabolic profile of these NCK1 KO animals were compared to WT. Mass spectrometry analysis (LC/MS) of blood samples revealed the expression of multiple molecules involved in biochemistry pathways of muscle regeneration and growth in this preclinical animal model. To validate these findings we exercised the animals for 4 weeks using treadmill training. The first week they were acclimated to moderate speed at 20 cm/s and the following weeks the speed was increased by 2-3 cm/s up to 28 cm/s at the final week. The mice run on the treadmill for 30 min for 5 days per week. At the end of the 4 week period, the blood was collected and subjected to analysis. The blood was centrifuged for serum separation. Serum samples were used in mass spectrometry analysis. For sample preparation, serum was diluted using methanol. Then the samples were centrifuged for 5 min at 10000 rpm. Supernatant was removed and diluted to 1:100 using HILIC buffer.

Samples were centrifuged again for 5 min at 10000 rpm and supernatant were added to MS vials. Mass spectrometry were performed in duplicate. Serum of at least three different animals were used in this experiment. Data were collected and analyzed using Compound Discoverer (Thermo Scientific).

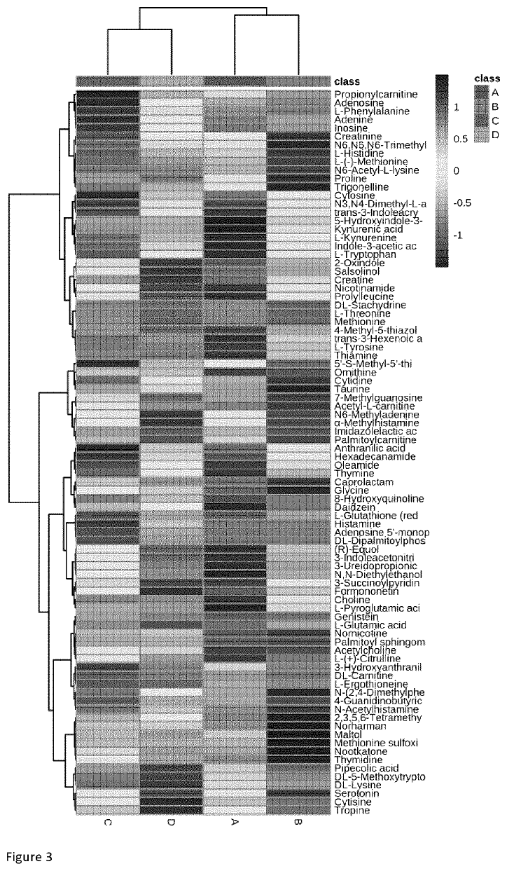

We identified a subset of molecules that returned to control levels in response to exercise routine (FIG. 3). Groups are as followed: A—WT sedentary, B—NCK1 KO sedentary, C—WT exercised and D—NCK1 KO exercised.

Taurine is a amino acid involved in the catabolism of muscle directed by protein metabolism, this amino acid was altered before exercised and returned to WT levels post exercise routine. Taurine has been shown to be involved in mitochondria bioenergetic signalling and reduced levels of taurine is linked to aging associated conditions such as cardiovascular disease and skeletal muscle disorders.

Creatinine levels are correlated with muscle mass. In here, we observed a 1.7-fold increase in NCK1KO compared to WT. Creatinine ratios after exercise routine decreased to 1.1-fold, shifting metabolism to muscle anabolism are shown in Table I.

TABLE I

| | | | | | WT 3 m | NCK1KO | | |
|---|---|---|---|---|---|---|---|---|
| Metabolites | WT 3 m | NCK1KO | | p- | (post | 3 m (post | | p- |
| in Blood | old | 3 m old | Ratio | value | exercise) | exercise) | Ratio | value |
| Taurine | 7190921.655 | 3713740.45 | 0.51 | *0.01 | 6792192.25 | 4527216.362 | 0.7 | 0.1 |
| Citrulline | 302304.2118 | 1252361.657 | 4.1 | *0.004 | 418893.6 | 903377.0 | 2.1 | *0.09 |
| Ornithine | 1232130.922 | 848625.09 | 0.68 | 0.13 | 1154768.439 | 1069066.183 | 0.9 | 0.2 |
| Creatinine | 478783.651 | 802617.8694 | 1.7 | *0.009 | 365896.936 | 404190.5082 | 1.1 | 0.6 |
| Maltol | 12302.81126 | 17559.9247 | 1.4 | *0.004 | 12582.70381 | 12833.24976 | 1.0 | 0.9 |
| Trigonelline | 1835271.569 | 2831396.326 | 1.5 | *0.03 | 1629376.57 | 1754216.45 | 1.1 | 0.9 |
| Thymidine | 17789 | 33485 | 1.8 | 0.1 | 19959.2 | 25908.45 | 1.3 | 0.6 |

We also observed trigonelline increase (1.5-fold). Trigonelline was showed to have a protective effective during muscle loss by increasing insulin sensitivity in soleus muscle, this increase induces muscle growth under disuse conditions. Elevation of trigonelline could be linked to a feedback loop to prevent more loss of muscle myofibers. After exercise, level of trigonelline returned to WT levels.

Thymidine a nucleoside involved in DNA synthesis was also found elevated (1.8-fold) in NCK1 KO compared to WT. Thymidine is involved in myogenesis and is correlated with the proliferation of precursors myoblast cells that are activated upon injury or damage of skeletal muscle. The increase in thymidine as between WT versus NCK1 KO may be implicated in muscle stem cells activation that are stimulate upon damage of fibers. After exercise, the NCK1 KO levels of thymidine was slightly decreased. Hypertrophy of fibers is promoted by exercise and protein synthesis therefore a higher number of cell nuclei increases protein synthesis efficacy. Without being limited to any particular theory, it is possible that once cell metabolism is rolling to protein synthesis, DNA synthesis is decreased which caused the levels of thymidine in NCK1 KO to decrease and be more similar to WT levels.

Maltol was increased in NCK1 KO sedentary animals. Maltol has been shown to play a role in controlling glucose levels in cells resistant to insulin. Maltol also decreased inflammation in a model of osteoarthritis. Without being limited to any particular theory, the effect of maltol on NCK1 KO may be a compensatory mechanism to improve protein synthesis and reduce skeletal muscle damage. We observed significant increase of maltol in NCK1 KO sedentary animals and after exercise the level decrease to WT levels.

From the analysis of blood samples of animals before and after exercise we identified a panel of metabolites that were altered in NCK1 KO animals and, after exercise, these metabolites returned to WT levels. We found key clusters of amino acids, metabolites and neurotransmitters that may be an interesting set of biomolecules to correlate with muscle atrophy. From the present analysis, it is clear that major hallmarks of muscle atrophy may be established before condition like sarcopenia started to develop and therefore, the presence of the identified panel of biomarkers may be an early indicator of aging-related disorders.

These results demonstrate a strong correlation between molecular biomarkers and recovery state or healthy state of each individual animal and confirm a functional link between biomarker levels and muscle atrophy. In addition, we identified specific metabolomics pathways and bioenergetics metabolites that were altered.

### Example 2—Human Samples—Serum Analysis

To further validate the animal model observations, we analyzed the presence of these metabolites in human samples. Notably, we were interested in glutamate, taurine and nicotinamide metabolism. In human samples we observed differences in metabolites involved in the urea cycle that is linked to glutamate metabolism and also observed differences in taurine. Additionally, we were interested in analyzing female metabolism as the animal study was completed in males only.

For these studies, human serum samples were divided in 8 groups. Groups A, B, C and D were composed by serum collected from males and groups E, F, G and H were composed by female serum. Group A/E were composed by individuals aged 35-45 years old and physically active. Physical activity status was measured using the International physical activity questionnaire (IPAQ) and scored at high and low. Group B/F was composed by individual age 50-70 years old and physically active (high IPAQ). Group C/G was composed by individuals aged 50-70 years old and sedentary (low IPAQ). Group D/H was composed by individuals aged 50-70 years old, sedentary, and diagnosed with arthritis and/or osteoporosis.

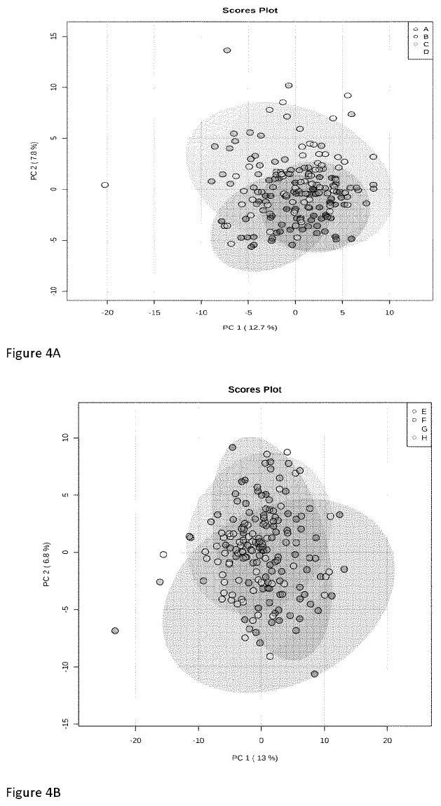

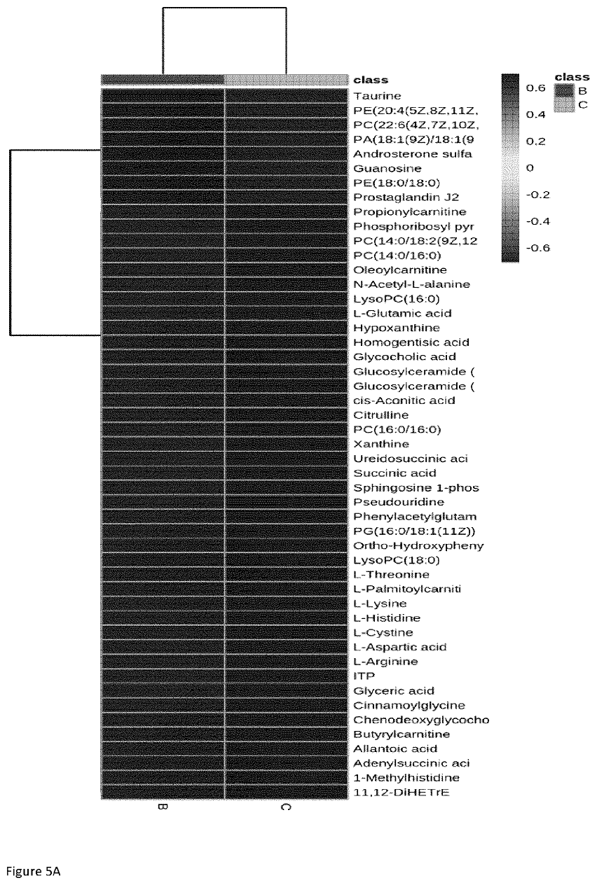

In the male groups, we found that individuals on group B, C and D displayed differences in metabolites expression compared to group A. Group C and D displayed similar metabolites profile (FIG. 4A). Group B and C displayed differences that were similar with our observation in the animal models. Of the total number of metabolites, 44 displayed statistical significance between group B and C (FIGS. 5A and B). Among other metabolites demonstrating a significant presence and variation, the presence and variation of L-aspartic acid, L-arginine, L-glutamic acid, homogentisic acid, taurine and methylhistidine is notable.

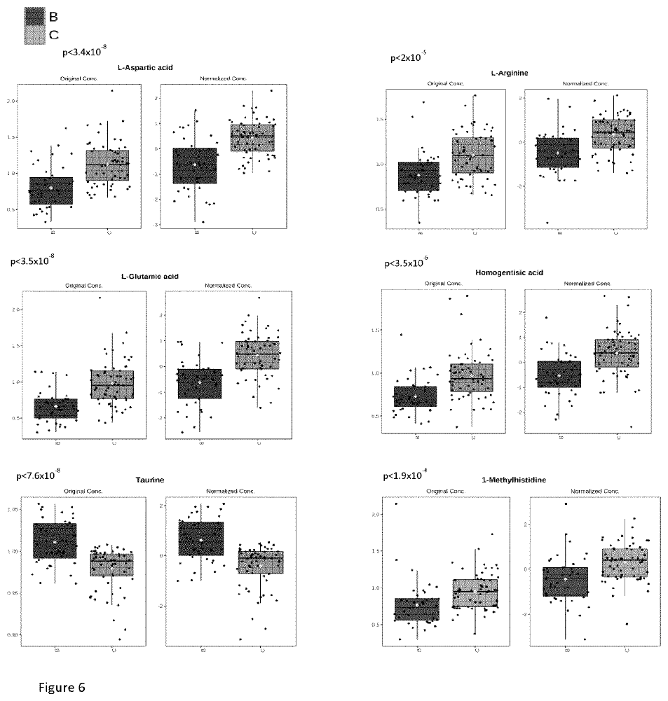

In the female groups we observed that all the groups were similar (FIG. 4B), however when we narrowed the analysis to group F and G (FIG. 7A, B) we observed a pattern. Several of the metabolites identified were different from the metabolites in males. This was expected since males and females muscle metabolism are very different. However, we discovered that some of the metabolites in glutamate and urea cycle are significantly different between the groups, similarly to what we observed in males. Among other metabolites demonstrating a significant presence and variation, the presence and variation of L-arginine, L-glutamine, citrulline, methylhistidine and oxoglutaric acid is notable.

### Example 3—Human Samples—Urinalysis

Understanding metabolic adaptations in urine facilitates metabolite monitoring on a regular basis.

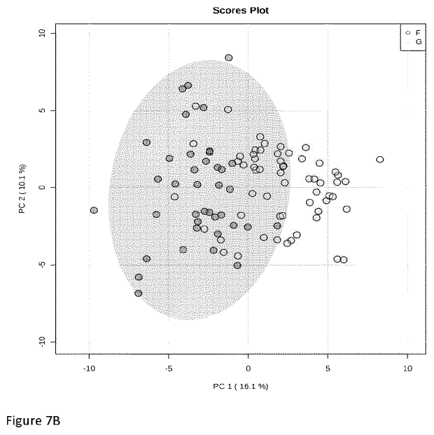

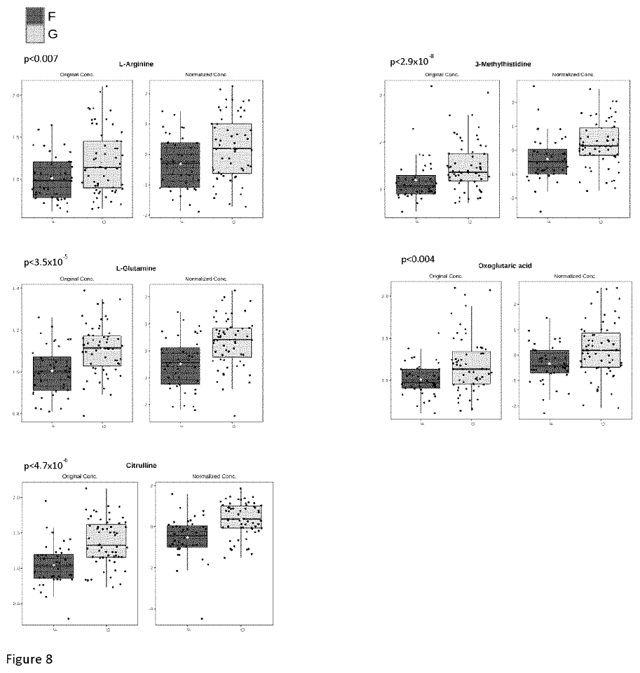

FIGS. 9 and 10 shows the results of urine analysis of a panel of metabolites comparing exercise vs sedentary subjects with a sample size of n=80. As shown in FIG. 9, the presence and variation of metabolites in females and males responded differently. Individually, the change in metabolites were mostly significant in females and the change trended consistently in males.

To confirm the relationship between metabolite levels and physical activity, a receiver operating characteristic curve (ROC) was created (FIG. 10). Through the application of linear regression analysis on urinalysis data, it was revealed that metabolites taurine, xanthine, L-carnitine, succinate and glutamate strongly correlated with physical activity levels in both male and female participants, where the area under the curve (AUC) was 0.8 and 0.96 for males and females, respectively. These results revealed significant associations between the level of these metabolite and physical activity parameters.

### Example 4—Clinical Study to Assess Relationship of Biomarkers Panel with Muscle Mass, Strength, and Function

During muscle atrophy, regeneration, and/or recovery, the molecular changes occur much quicker than physically observable changes. Therefore, identification and characterization of these molecular biomarkers benefits rehabilitation programs and therapeutical regimen.

A clinical study was conducted to confirm biomarkers of muscle atrophy establishing muscle health for patients and as evidence-based model to improve individuals physical health and wellness. The obtained biological samples allow correlation of specific biomarkers with functional and overall muscle mass.

During muscle regeneration and recovery, molecular changes occur much quicker than physically observable changes. The identification of these molecular biomarkers will benefit rehabilitation programs and therapeutical regimen.

In this scenario, acquiring more knowledge into molecular changes will provide valuable insights into patient specific care and recovery plans.

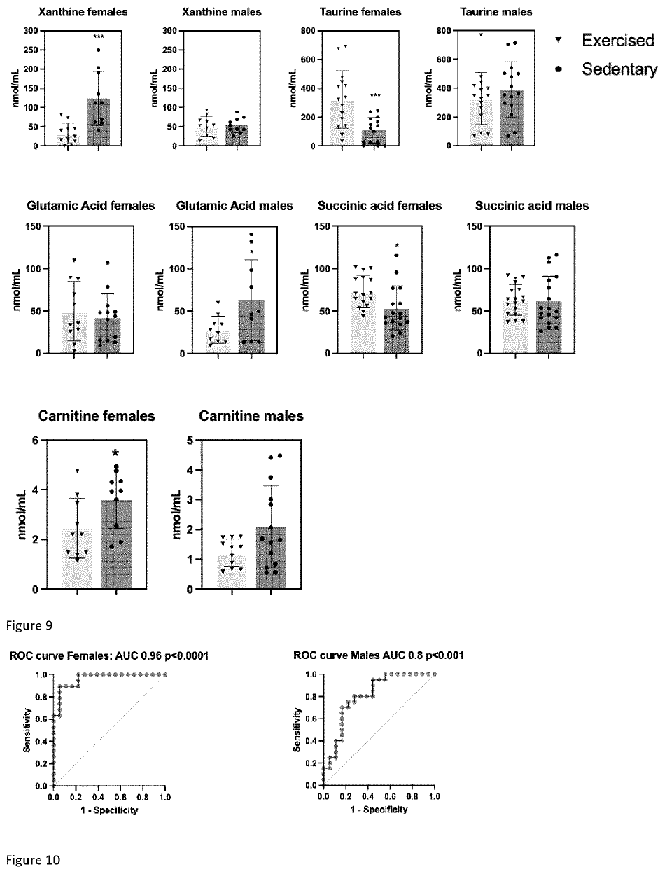

An overview of the study is shown in FIG. 11 where 60 participants, 30 males and 30 females between 50-70 years old were recruited to undergo an extensive physical assessment by measuring grip strength, time up and go, sit and stand, gait speed, timed stance tests and determined the overall short physical performance battery (SPPB) score to monitor physical performance and function.

In addition, we conducted dual x-ray absorptiometry (DXA) imaging and determine the appendicular lean mass (ALM). The DXA score was normalized against height to all participants using the formula appendicular lean mass index (ALM)/(height)². We determined overall SPPB and DXA scores using standardized methods of reference. The ALMI and SPPB represent an objective screening tool to diagnose sarcopenia as established by the European Working Group on Sarcopenia in Older People (EWGSOP). A cut off lower than 6 for SPPB has a high specificity and sensitivity based on EWGSOP criteria. For DXA, males and females have different cut offs, <5.67 kg/m² and <7.26 kg/m², respectively and for 5× sit and stand scores lower than 14 seconds are indicative of sarcopenia.

Muscle health is considered to be a combination of total muscle mass, performance and functional assessments and therefore to conduct a more comprehensive analysis of muscle health we defined five categories to identify various levels of muscle health and assigned a score depending on the association with sarcopenia: sarcopenic, pre-sarcopenic, low muscle health, intermediate and ideal. These categories received a numeric weight (Table 2). The cut off value of 2≥ was scored as high muscle health and 2< was scored as low muscle health.

TABLE 2

| | Ranges | | | | |
|---|---|---|---|---|---|
| | | Pre- | Low muscle | Intermediate muscle | Ideal muscle |
| | Sarcopenic | sarcopenic | health | health | health |
| Weights | 0 | 1 | 1.5 | 2 | 2.5 |
| ALMI | <7.26 kg/m2 | 7.27-8.21 | 8.22-9.43 | 9.44-10.28 | 10.29> |
| Females | | | | | |
| ALMI | <5.67 kg/m2 | 5.68-6.40 | 6.41-7.36 | 7.37-8.02 | 8.03> |
| Males | | | | | |
| SPPB | <6 | 7 | 8 | 9 | 10> |
| 5X | >14 | 12.1-14 | 11.1-12 | 10-11 | <10 |
| sit/stand | | | | | |

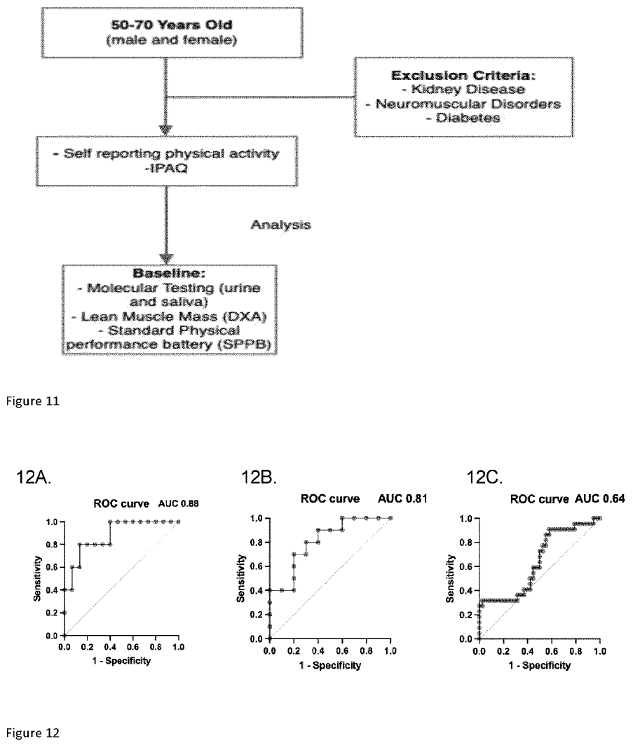

Samples of urine were collected. We correlated each one of these metrics with our metabolic data to show the accuracy of the biomarker panel comprising taurine, xanthine, L-carnitine, succinate and glutamate compared to the standard muscle health assessments. We found that ALMI and 5× sit and stand produce the highest AUC of 0.88 (FIG. 12A). When combining SPPB with ALMI obtained an AUC score of 0.81 (FIG. 12B) and combining 5× Sit and stand with SPPB we obtained an AUC score of 0.68 (FIG. 12C).

The example shows that the biomarker panel is useful in providing the status of muscle health and inform the development of effective interventions and treatments.

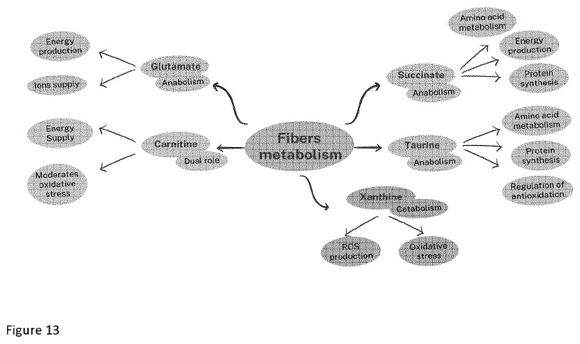

As shown in FIG. 13, the present example highlights the interconnectedness of muscle function, muscle mass, and muscle metabolism. The variation of 5 metabolites: taurine, xanthine, L-carnitine, succinate and glutamate predicts overall muscle health. Their presence and variation in urine samples is linked to fibers metabolism that directly impacts overall muscle health. The observed correlations suggest that optimal muscle function is closely associated with greater muscle mass and favorable metabolic profiles. The findings also showed the significance of muscle mass and functional analysis in influencing muscle metabolism and overall muscle health. These results establish a correlation between the presence and variation of specific biomarkers with muscle health as analyzed by these parameters.

In conclusion, early detection of biomarkers of muscle health has the potential to improve early diagnosis and monitoring of muscle-related diseases. While there is no cure for most of these illnesses, the correlation of biomarkers in bodily fluids such as in serum and in urine with prognosis and disease progression will help in monitoring treatment outcomes.

### Example 6—Clinical Study to Assess Prognosis Information Based on Muscle Atrophy Levels for Improvement of Physical Health, Triage of Patients and Recovery Efficiency

Assessment levels of muscle atrophy for patients entering physical training programs or pre-rehabilitation to confirm a correlation with improvement of muscle health and recovery capability.

Participants enter a rehabilitation program, half of patients will start a specialized rehabilitation program while the other half will continue to standard rehabilitation care. In the specialized rehabilitation program, the patients will go through a 6 week recovery training at the gym to rebuild muscle strength and mass. They will attend 30 minutes targeted fitness class 4× per week, where they will be focusing on lower limb strengthening exercises and mobility. Typical movements are squats, lunges, deadlifts, pushups and staggered stance or single leg/arm workout to help with balance. While in the program, participants undergo physical assessments and blood (every 2 weeks) and urine/saliva collection every week. After the 6-weeks training participants in both groups will return to hospital for clinical assessment. Common physical assessments are based on gait, strength, and balance tests. These parameters are correlated with recovery and are the golden standards of recovery assessments. We will be performing timed up and go, 30-sec chair stand and 4-stage balance tests. At the end of the recovery training, muscle mass will be assessed by DXA to confirm recovery status in comparison to the first scans prior to initiate the program.

Physical assessments will be accomplished by the completion of physical tasks. Muscle strength will be measured, joint range motion and balance. We will later compare the results of physical assessments with biomarkers levels throughout the program. Samples of blood, urine and saliva will be used to identify biomarkers and quantify levels. This information will enable confirmation of reversion of biomarkers throughout the exercise program and will be utilized to corroborate the level of biomarkers and final recovery status for prognosis information, assess the risk of complication, and recovery progress.

In some embodiments one or more biomarkers and/or a panel of biomarkers of the present disclosure as provided in Table 3 below provides information on prognosis, muscle health and performance and a correlation with level of muscle atrophy. The analysis provides an accurate and timely reflection of disease severity and patient specific muscle health, predicting to health care providers and individuals their muscle atrophy status. The present disclosure provides an objective measurement of muscle atrophy and will identify patients who are at risk of muscle related conditions such as osteoarthritis so that specific interventions can be made to physical routine, diet and therapy regimen. Finally, the objective measurement of muscle atrophy provides justification for preventive therapy and support muscle health maintenance. Information on muscle health can support pre-rehabilitation programs, targeting reversible stages of musculoskeletal conditions when physical deterioration cannot be conclusive.

TABLE 3

| | | |
|---|---|---|
| 1-Methylhistidine | Glyceric acid | Norharman |
| (R)-Equol | Glycerophosphocholine | Nornicotine |
| 11,12-DIHETrE | Glycine | Oleamide |
| 1 -Methylnicotinamide | Glycocholic acid | Oleoylcarnitine |
| 2,3,5,6-Tetramethylpyrazine | Guanidoacetic acid | Ornithine |
| 2-Isopropylmalic acid | Guanosine | Ortho-Hydroxyphenylacetic acid |
| 2-Octenoylcarnitine | Hexadecanamide | Oxoglutaric acid |
| 2-Oxindole | Histamine | PA(16:0/16:0) |
| 3,5-Tetradecadiencarnitine | Homogentisic acid | PA(18:1(9Z)/18:1(9Z)) |
| 3-Hydroxyanthranilic acid | Homo-L-arginine | Palmitoyl sphingomyelin |
| 3-Indoleacetonitrile | Hydroxyhexanoycarnitine | Palmitoylcarnitine |
| 3-Methylhistidine | Hypotaurine | Pantothenic acid |
| 3-Succinoylpyridine | Hypoxanthine | PC(14:0/16:0) |
| 3-Ureidopropionic acid | Imidazolelactic acid | PC(14:0/18:2(9Z, 12Z)) |
| 4-Guanidinobutyric acid | Indole-3-acetic acid | PC(16:0/16:0) |
| 4-Hydroxyproline | Indoleacetic acid | PC(16:0/16:0) |
| 4-Methyl-5-thiazoleethanol | Indoxyl sulfate | PC(22:6(4Z, 7Z, 10Z, 13Z, 16Z, 19Z)/P- |
| | | 18:0) |
| 5-Hydroxyindole-3-acetic acid | Inosine | PE(18:0/18:0) |
| 5′-Methylthioadenosine | Isocitric acid | PE(20:4(5Z, 8Z, 11Z, 14Z)/18:1(9Z)) |
| 5′-S-Methyl-5′-thioadenosine | ITP | PG(16:0/18:1(11Z)) |
| 7-Methylguanosine | Kynurenic acid | Phenylacetylglutamine |
| 8-Hydroxyquinoline | L-(−)-Methionine | Phenylpyruvic acid |
| 9-Decenoylcarnitine | L-(+)-Citrulline | Phosphoribosyl pyrophosphate |
| Acetylcholine | L-Acetylcarnitine | Phosphorylcholine |
| Acetylcholine chloride | L-Arginine | p-Hydroxyphenylacetic acid |
| Acetyl-L-carnitine | L-Aspartic acid | Pimelylcarnitine |
| Adenine | L-Cystine | Pipecolic acid |
| Adenosine | L-Ergothioneine | Proline |
| Adenosine 5′-monophosphate | L-Glutamic acid | Prolylleucine |
| Adenylsuccinic acid | L-Glutamine | Propionylcarnitine |
| Allantoic acid | L-Glutathione (reduced) | Prostaglandin D2 |
| Allantoin | L-Histidine | Prostaglandin J2 |
| Androsterone sulfate | L-Kynurenine | PS(16:0/16:0) |
| Anthranilic acid | L-Lactic acid | PS(18:0/18:1(9Z)) |
| Asymmetric dimethylarginine | L-Lysine | Pseudouridine |
| Betaine aldehyde | L-Palmitoylcarnitine | Pyridoxal 5′-phosphate |
| Butyrylcarnitine | L-Phenylalanine | Pyroglutamic acid |
| Caprolactam | L-Pyroglutamic acid | Salsolinol |
| Chenodeoxyglycocholic acid | L-Serine | Serotonin |
| Choline | L-Threonine | S-Methyl-L-cysteine |
| Cinnamoylglycine | L-Tryptophan | Sphingosine 1-phosphate |
| cis-Aconitic acid | L-Tyrosine | Stearoylcarnitine |
| Citrulline | LysoPC(16:0) | Suberic acid |
| Creatine | LysoPC(18:0) | Succinic acid |
| Creatinine | LysoPC(24:1(15Z)) | Taurine |
| Cysteamine | Maltol | Testosterone |
| Cytidine | Metanephrine | Tetradecanoylcarnitine |
| Cytisine | Methionine | Thiamine |
| Cytosine | Methionine sulfoxide | Thromboxane B2 |
| Daidzein | N-(2,4- | Thymidine |
| | Dimethylphenyl)formamide | |
| DL-2-Aminooctanoic acid | N,N-Diethylethanolamine | Thymine |
| DL-5-Methoxytryptophan | N3,N4-Dimethyl-L-arginine | Tiglylcarnitine |
| DL-Carnitine | N6,N6,N6-Trimethyl-L- | trans-2-Dodecenoylcarnitine |
| | lysine | |
| DL-Dipalmitoylphosphatidylcholine | N6-Acetyl-L-lysine | trans-3-Hexenoic acid |
| DL-Lysine | N6-Methyladenine | trans-3-Indoleacrylic acid |
| DL-Stachydrine | N-a-Acetyl-L-arginine | Trigonelline |
| D-Ribose 5-phosphate | N-Acetylhistamine | Tropine |
| D-Ribulose 5-phosphate | N-Acetyl-L-alanine | Ureidosuccinic acid |
| Formononetin | N-Acetylmannosamine | Uric acid |
| Genistein | N-Acetylserine | Uridine |
| Glucosylceramide (d18:1/16:0) | Nicotinamide | Xanthine |
| Glucosylceramide | Nootkatone | Xylulose 5-phosphate |
| (d18:1/24:1(15Z)) | | |
| | | α-Methylhistamine |

The embodiments of the present application described above are intended to be examples only. Those of skill in the art may effect alterations, modifications and variations to the particular embodiments without departing from the intended scope of the present application. In particular, features from one or more of the above-described embodiments may be selected to create alternate embodiments comprised of a subcombination of features which may not be explicitly described above. In addition, features from one or more of the above-described embodiments may be selected and combined to create alternate embodiments comprised of a combination of features which may not be explicitly described above. Features suitable for such combinations and subcombinations would be readily apparent to persons skilled in the art upon review of the present application as a whole. Any dimensions provided in the drawings are provided for illustrative purposes only and are not intended to be limiting on the scope of the invention. The subject matter described herein and in the recited claims intends to cover and embrace all suitable changes in technology.

1. A method for diagnosing a subject with early onset muscle atrophy, said method comprising:
analyzing a biosample obtained from the subject for a panel comprising three or more biomarkers, said biomarkers selected from taurine, proline, citrulline, trigonelline, thymidine, ornithine, glutamate, L-pyroglutamic acid, creatinine, adenine, nicotinamide, 2-methylhippuric acid, maltol, L-arginine, hypotaurine, L-glutamine, homogentisic acid, methylhistidine, oxoglutaric acid, xanthine, L-carnitine, or succinate to determine a subject value of said ene or mere biomarkers; and
diagnosing the subject with early onset muscle atrophy where there is a deviation in the subject value from a threshold value of said biomarkers.

2. The method of claim 1 wherein the biosample is from blood, saliva, or urine.

3. The method of claim 1 wherein the biosample is urine and said panel comprises three or more biomarkers, said biomarkers selected from taurine, xanthine, L-carnitine, succinate and glutamate or said panel consists essentially of taurine, xanthine, L-carnitine, succinate and glutamate; or wherein the biosample is blood and said panel comprises three or more biomarkers, said biomarkers selected from L-aspartic acid, L-arginine, L-glutamine, L-glutamic acid, taurine, homogentisic acid, citrulline, methylhistidine and oxoglutaric acid; or said panel consists essentially of L-aspartic acid, L-arginine, L-glutamic acid, homogentisic acid, taurine and methylhistidine; or said panel consists essentially of L-arginine, L-glutamine, citrulline, methylhistidine and oxoglutaric acid.

4. The method of claim 1 wherein said panel comprises glutamate, xanthine, taurine, succinate, and L-carnitine.

5. The method of claim 4 wherein the biosample is urine.

6. The method of claim 1 wherein the subject alue is a composite of the panel comprising three or more biomarkers and the threshold value is from a population of normal subjects representing the 75^{th}, 85^{th}, 90^{th}, 95^{th}, or 99^{th} percentile of the biomarker as measured in said normal subjects.

7. (canceled)

8. (canceled)

9. (canceled)

10. (canceled)

11. (canceled)

12. (canceled)

13. A method of maintaining muscle health of a subject, the method comprising:
obtaining a biosample from the subject;
assaying the biosample for a panel comprising three or more biomarkers, said biomarkers selected from taurine, proline, citrulline, trigonelline, thymidine, ornithine, glutamate, L-pyroglutamic acid, creatinine, adenine, nicotinamide, 2-methylhippuric acid, maltol, L-arginine, hypotaurine, L-glutamine, homogentisic acid, methylhistidine, oxoglutaric acid, xanthine, L-carnitine, or succinate to determine a subject value of said biomarkers;
administering an exercise regimen to the subject to maintain muscle health;
obtaining a further biosample from the subject after the exercise regimen;
assaying the further biosample to determine a post exercise value of said one or more biomarkers;
comparing the post exercise value for said one or more biomarkers in the further biosample to a threshold value; and
administering any further exercise regimen when the post exercise value is different than the threshold value.

14. The method of claim 13 wherein the biosample is from, blood, saliva or urine.

15. The method of claim 14 wherein the biosample is urine and said panel comprises three or more biomarkers selected from taurine, xanthine, L-carnitine, succinate or glutamate or said panel consists essentially of taurine, xanthine, L-carnitine, succinate and glutamate is taurine, xanthine, L-carnitine, succinate and glutamate; or wherein the biosample is blood and said panel comprises three or more biomarkers selected from L-aspartic acid, L-arginine, L-glutamine, L-glutamic acid, taurine, homogentisic acid, citrulline, methylhistidine or oxoglutaric acid; or said panel consists essentially of L-aspartic acid, L-arginine, L-glutamic acid, homogentisic acid, taurine and methylhistidine; or said panel consists essentially of L-arginine, L-glutamine, citrulline, methylhistidine and oxoglutaric acid.

16. The method of claim 13 wherein the panel comprises glutamate, xanthine, taurine, succinate, and L-carnitine.

17. The method of claim 13 wherein the subject value is a composite of the panel comprising three or more biomarkers and the threshold value is a composite from the panel comprising three or more biomarkers from a population of normal subjects representing the 75^{th}, 85^{th}, 90^{th}, 95^{th}, or 99^{th} percentile as measured in said normal subjects.

18. A method to treat muscle atrophy in a subject or to guide muscle recovery of a subject suspected of having a neuromuscular disease or after an orthopedic procedure, the method comprising:
analyzing a biosample for a panel comprising three or more biomarkers, said biomarkers selected from taurine, proline, citrulline, trigonelline, thymidine, ornithine, glutamate, L-pyroglutamic acid, creatinine, adenine, nicotinamide, 2-methylhippuric acid, maltol, L-arginine, hypotaurine, L-glutamine, homogentisic acid, methylhistidine, oxoglutaric acid, xanthine, L-carnitine, or succinate, to determine a subject value of said biomarkers in the subject;
identifying a deviation in the subject value from a threshold value;
administering a treatment regimen selected for increasing muscle growth;
analyzing a further biosample for said panel during or after the treatment regimen; and
identifying a treated subject or a recovered subject when there is no longer a deviation in the subject value from the threshold value of said biomarkers.

19. The method of claim 18 wherein the treatment regimen is physical exercise.

20. The method of claim 18 wherein the biosample is from blood, saliva or urine.

21. The method of claim 20 wherein the biosample is urine and said panel is three or more biomarkers selected from taurine, xanthine, L-carnitine, succinate or glutamate or said panel consists essentially of taurine, xanthine, L-carnitine, succinate and glutamate is taurine, xanthine, L-carnitine, succinate and glutamate; or wherein the biosample is blood and said panel comprises three or more biomarkers selected from L-aspartic acid, L-arginine, L-glutamine, L-glutamic acid, taurine, homogentisic acid, citrulline, methylhistidine or oxoglutaric acid; or said panel consists essentially of L-aspartic acid, L-arginine, L-glutamic acid, homogentisic acid, taurine and methylhistidine; or said panel consists essentially of L-arginine, L-glutamine, citrulline, methylhistidine and oxoglutaric acid.

22. The method of claim 20 wherein the panel comprises glutamate, xanthine, taurine, succinate, and L-carnitine.

23. The method of claim 20 wherein the subject value is a composite of the panel comprising three or more biomarkers and the threshold value is a composite from the panel comprising three or more biomarkers from a population of normal subjects representing the 75^{th}, 85^{th} 90^{th}, 95^{th}, or 99^{th} percentile as measured in said normal subjects.

24. (canceled)

25. (canceled)

26. (canceled)

27. The method of claim 13 where in the biosample is urine.

28. The method of claim 18 wherein the biosample is urine.
